(19) United States
(12) Patent Application Publication
(10) Pub. No.: US 2019/0116474 A1
(43) Pub. Date: Apr. 18, 2019

(54) A SENSOR NETWORK AND APPARATUS THEREFOR

(71) Applicants: George DRIDAN, McLaren Vale (AU); Charles Keith THOMPSON, Belair (AU)

(72) Inventors: George DRIDAN, McLaren Vale (AU); Charles Keith THOMPSON, Belair (AU)

(21) Appl. No.: 16/090,719

(22) PCT Filed: Apr. 27, 2017
(86) PCT No.: PCT/AU2017/050381

§ 371 Date: Oct. 2, 2018

(30) Foreign Application Priority Data
Apr. 28, 2016 (AU) 2016901559

(51) Int. Cl. H04W 4/38; G01N 33/24; H04Q 9/02; H04B 13/02

(52) U.S. Cl. CPC H04W 4/38; G01N 33/24; H04Q 2209/43; H04B 13/02; H04Q 9/02

(57) ABSTRACT

The present invention relates generally to the field of soil monitoring systems and in particular a networked wireless data logging system. In one form the invention comprises a sensor network for measuring at least one parameter within a soil body, including at least one first node comprising a subsurface communication module for connection to a first sensor, at least one second node comprising a surface communication module for connection to a second sensor, wherein both said subsurface and surface communication modules being in communication with each other via at least one first network, and wherein said subsurface communication module or said surface communication module acting as a gateway unit for communication with a computing device or processor via a second network.

### FIELD OF THE INVENTION

The present invention relates generally to the field of soil monitoring systems and in particular a networked wireless data logging system. In one form the present invention relates to a communication module for attachment to a sensor array wherein the communication module can be located above or below a ground surface.

### BACKGROUND OF THE INVENTION

Sensor networks are known in the prior art and typically include a number of individual nodes that are linked to a central processing unit (CPU). Some sensor networks are wired and connected to a gateway unit that is then connected by a Wide Area Network (WAN) to the CPU. These wired systems are however expensive to install and can be easily disrupted by damage to interconnecting cables.

Other sensor networks disclosed in the prior art comprise a wireless sensor network having a plurality of individual nodes. Each individual node includes a sensor or sensors, radio frequency (RF) transceivers, antenna and power source. The antenna is located above the ground surface and in some situations a separate cabled antenna is attached to an individual node to improve communication within the network.

Typically, both wired and wireless sensor networks are in communication with a CPU that is located off-site and can be used to monitor factors such as, but not limited to, soil moisture, temperature and humidity. The CPU may also be configured to control on-site systems where the sensor, or sensor(s), is/are located, such as an irrigation system.

Recent advances in electromagnetic (EM) sensor technology have made it possible to effectively undertake subsurface measurement of soil water. However, communication within a wireless sensor network using subsurface sensors has been found to be problematic and requires at least a portion of the antenna to extend above the ground surface or be at least at ground level.

Some subsurface sensors have a cover plate that is positioned at the surface of soil, however these can present a slipping hazard if used, for instance, in a sporting environment.

Remote data collection from widely distributed sensor networks has many environmental, commercial, and agronomic applications. However existing RF data loggers and low cost devices typically do not have the range, power management, accuracy or resolution required for high grade sensors.

It should be appreciated that any discussion of the prior art throughout the specification is included solely for the purpose of providing a context for the present invention and should in no way be considered as an admission that such prior art was widely known or formed part of the common general knowledge in the field as it existed before the priority date of the application.

The terms ‘ground level’ or ‘ground surface’ used throughout the specification refers to the surface of the soil profile in which the sensor is or can be located. The terms ‘parameter’ or ‘soil parameter’ used throughout the specification refers to a condition within the soil body that can be measured using a sensor, such as but not limited to, moisture, temperature, humidity, nutrient levels, salinity (current) and/or pH. Where the context allows the terms “sensor node” or “sensing node” may be used to describe the assembled transmitter, antenna, interface, sensor and battery. The term “gateway unit” used throughout the specification refers to a communication module that facilitates communication with a CPU.

### SUMMARY OF THE INVENTION

It could be broadly understood that the present invention relates generally to a node comprising a communication module for attachment to at least one sensor configured to measure a parameter or parameters within a soil body, wherein the communication module can be positioned above or below a ground surface and be connected wirelessly to a network of nodes, each comprising a respective communication module and at least one sensor.

In one aspect of the invention, but not necessarily the broadest or only aspect, there is proposed a sensor network for measuring at least one parameter within a soil body, including: at least one first node comprising a subsurface communication module for connection to a first sensor; at least one second node comprising a surface communication module for connection to a second sensor; wherein both said subsurface and surface communication modules being in communication with each other via at least one first network, and wherein said subsurface communication module or said surface communication module acting as a gateway unit for communication with a computing device or processor via a second network.

In one form the first network comprises a subsurface network, surface network and a local area network. The subsurface network being formed between a plurality of first nodes, the surface network being formed between a plurality of second nodes and the local area network being formed between the first and second nodes.

Preferably the configuration of the subsurface communication module and surface communication module are substantially identical, wherein a communication module can be either located above or below a ground surface. In another form the surface and subsurface communication modules have different configurations.

Preferably the first and second sensors are used to measure the same parameter, such as but not limited to soil moisture. Alternatively, the first and second sensors are different types and are used to measure different parameters. In still another form a secondary sensor can be connected to the communication module by way of a cable. The parameter(s) may be selected from a group containing: soil moisture, nutrients, temperature, humidity, flow rates, pressure, voltage, current, salinity, or any other parameter used to determine a condition within the soil body.

Preferably the first network is a wireless local area network and the second network is wide area network. In one form the wide area network is a Wireless Wide Area Network (WWAN) using standard protocols, such as but not limited to, GSM (Global System for Mobile communication), GPRS (General Packet Radio Service), Satellite, UMTS (Universal Mobile Telecommunications System), or CDMA (Code Division Multiple Access).

In one form, each of the first and second nodes comprises a processor/interface, a communication module and a power source. The processor/interface, communication module and power source may be positioned within a housing that can be removably attached to a respective sensor. The processor/interface, communication module, power source and housing may at times be referred to generally as the communication module throughout the specification.

Each of the first and second nodes may include an auxiliary sensor/s for measuring a condition of said first or second sensor, or a condition of said communication module, or a condition of said power source. The auxiliary sensor therefore may be used to measure humidity within said sensor, active communication link or battery life.

The sensor network may be able to monitor existing hardware, collect data and package it in a format to integrate with existing GUI (Graphic User Interface) platforms.

The communication module can be installed in a subsurface configuration for discrete or application reasons. In one form the sensor node may be buried at a suitable depth wherein ploughing of a field can still occur without disturbance of the sensor node. The communication module can also be installed at surface level to allow for retro fitting to existing or new crop applications. The communication module may alternatively be installed as an elevated base or mesh repeater to expand the geographical scope of the network.

The sensor network may be set up as a mesh system or point to point system with a multipoint option to build in redundancy for control/monitoring networks.

The sensor network may be a monitoring and control network, wherein the system is able to both monitor a soil condition and control other onsite systems. For instance, the sensor network may be configured to control the operation of an irrigation pump (start/stop), valve control, and/or generator control.

The control function of the sensor network may include the ability to incorporate “SMS” (Short Message Service) monitoring and/or control.

The sensor network may also include self-learning capabilities for automatically controlling the other onsite systems. Furthermore, the communication module can be preloaded with data or data can be uploaded to the module, such as historic averages or predetermined values so that the sensor network can undertake certain actions when an event does or does not occur. The sensor network may also contain information on what water sources are available for utilization, e.g. bore, mains and/or dam, and will provide control of the pumps at each source.

In one form when connected to an irrigation system the sensor network will be able to determine what the normal water usage at a site should be, +/−1%-15% and preferably 5%. In another form when connected to a soil moisture probe the sensor network is able to determine the current status of the soil moisture and how long a typical irrigation will take to replenish the profile, +/−1%-15% and preferably 5%.

The sensor network may therefore be a simple sensor network providing information from the field to an end user or a measurement and control system that includes self-learning capabilities.

The power source in one form comprises an integrated battery/s being high capacity primary cells, having a shelf life of up to 10 years. By utilizing inductive charging and high capacity rechargeable batteries, the useful life and flexibility of the system can be extended even further. This also allows the system, under normal operation, to remain in situ (underground) for an extended period, for instance up to 8 years on a single battery pack. In one form the sensor network utilizes an integrated battery through a multi communication platform with power activated key technology.

Preferably a high power mesh network is used to optimize coverage and reliability and an in tandem micro network provides long life micro sensors.

Each of the communication modules within the sensor network preferably includes an antenna array having a radiating element and tuned ground planes. In one form the antenna incorporates a tuned circuit based on a ¼ ground wave layout. The antenna array includes a ground plane shield that keeps ground plane and coax in place and secure. A base plate provides a connection port for the antenna and magnetic reed switch and activation key.

The communication module housing may include a multi fit adaptor that allows connection to different types and configurations of sensors. The battery may be located within the housing or alternatively the battery unit may be connected to the housing wherein a portion of the battery unit is able to extend into an internal cavity of a sensor. Typically, the configuration of existing annular shaped sensors results in an internal cavity into which the battery can be inserted. This therefore reduces the overall height of the communication module which means that the sensor, with communication module attached, can be located in a position as close to the soil surface as possible, when the communication module is positioned in a subsurface arrangement.

The communication module may incorporate a 915 Mhz mesh radio with power requirements of 120 mA TX/30 mA RX and 2.5 uA sleep, and a micro mesh radio with power requirements of 31 mA TX/15 mA RX and <1 uA sleep. The communication module may provide 3G/4G/5G or satellite connectivity.

The communication module includes appropriate seals and closure to maintain a high level of ingress protection (IP). In one form the communication module IP once connected to an appropriate sensor has or exceeds IP67. Whilst the nodes will be sealed to meet the required IP rating, the design is preferably modular so that all components can be changed or serviced with minimal effort.

The short range local network may connect a plurality of sensors within 10-100 m of each other. It is envisaged that the local network may be an independent low power radio network, however other networks such as BLUETOOTH®, BLUETOOTH®Low Energy (BLE), or a network that permits a number of active connections are possible.

In another aspect of the invention there is proposed a method of communication between sensors located within, or adjacent, a soil body and a computing device, including the steps of: locating a plurality of first nodes in the soil body, each first node comprising a first communication module attached to a first sensor, wherein the respective first communication modules are concealed below a surface of said soil body; locating a plurality of second nodes in the soil body, each second node comprising a second communication module attached to a second sensor, wherein at least a portion of the respective second communication modules extend above the surface of the soil body; establishing a subsurface network between the plurality of first nodes; establishing a surface network between the plurality of second nodes; establishing a local area network between said first and second nodes; and establishing a wide area network between said first and/or second nodes and the computing device to transfer information from said first and second nodes to said computing device.

The subsurface network, surface network and local area network for a first network. In one form the first network comprises at least two sub-networks, wherein a plurality of first sensors create a subterranean sub-network and a plurality of second sensors create a surface sub-network.

The above method further includes the step of controlling a control system based upon the information received by said computing device. In one form the control system is an irrigation system for use in irrigating said soil profile.

In yet another aspect of the invention there is proposed a sensing node comprising a radio, antenna, interface, power supply and sensor/s, wherein the sensing node can be located in first position, wherein it is in a subterranean arrangement, or the sensing node can be located in second position wherein a portion thereof can extend above a ground surface, whereby in either first or second positions the sensing node is connectable to a single local area network.

In yet still another aspect of the invention there is proposed a communication module comprising a data transmission unit (DTU)/transceiver having a radio and antenna, and an interface and a power supply that are wired to a sensor array.

### BRIEF DESCRIPTION OF THE DRAWINGS

The accompanying drawings, which are incorporated in and constitute a part of this specification, illustrate an implementation of the invention and, together with the description and claims, serve to explain the advantages and principles of the invention. In the drawings,

FIG. 1 is a schematic view of a sensing node of the prior art;

FIG. 2 is a perspective view of an embodiment of the communication module of the present invention;

FIG. 3 is an exploded view of the communication module of FIG. 2;

FIG. 4 is a schematic view of the communication module attached to a sensor wherein the communication module/s are positioned in a subterranean location;

FIG. 5 is a schematic view of one embodiment of the sensor network;

FIG. 6 is a schematic view of a second embodiment of the sensor network illustrating communication modules positioned in subterranean and surface locations; and

FIG. 7 is a perspective view of another embodiment of the communication module of the present invention.

### DETAILED DESCRIPTION OF THE ILLUSTRATED AND EXEMPLIFIED EMBODIMENTS

Similar reference characters indicate corresponding parts throughout the drawings. Dimensions of certain parts shown in the drawings may have been modified and/or exaggerated for the purposes of clarity or illustration.

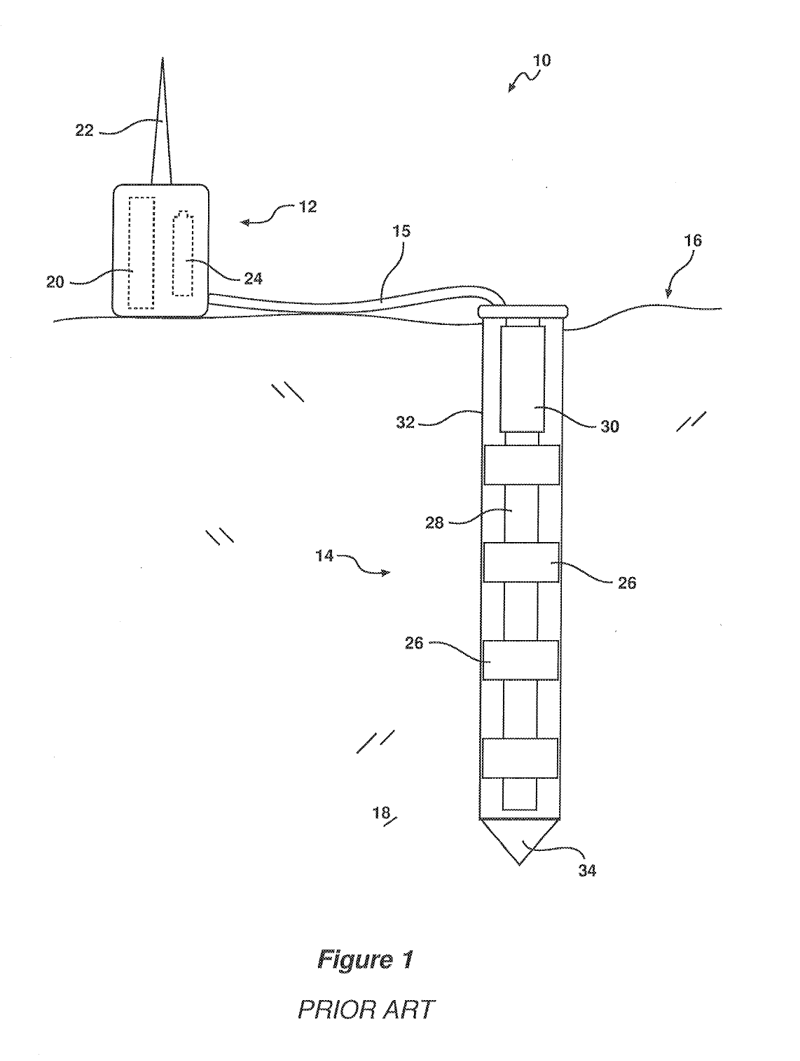

FIG. 1 illustrates an example of an existing hypothetical sensing node 10 currently used within the field. The sensing node 10 includes data transmission unit (DTA) 12 and a sensor array 14. The data transmission unit 12 is located at a distance from the sensor array 14 and connected by cable 15. This ensures that the data transmission unit 12 can be located in a position to permit contact with a wireless network. The data transmission unit 12 includes a processor/transmitter 20, external antenna 22 and battery 24. The sensor array 14 in the present example includes a plurality of sensors 26 attached along a rod 28, and a sensor interface 30, which are all located within an access tube 32 having a slurry bottom stopper 34 at a lower end thereof. The skilled addressee will however appreciate that the sensor array 14 could include a single sensor.

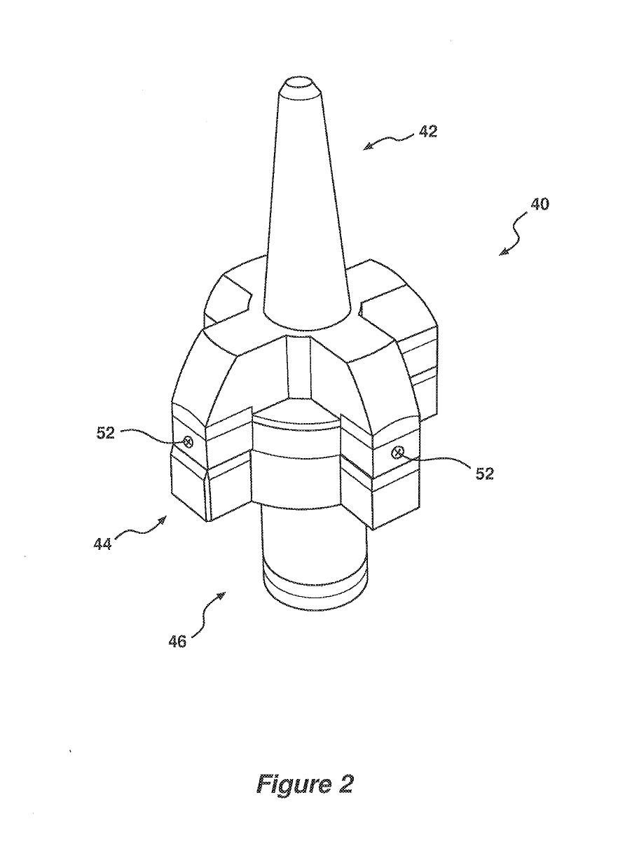

Referring to FIGS. 2 to 7 for a more detailed description of the present invention, a communication module 40 and sensor network 72 are illustrated, demonstrating by way of examples, arrangements in which the principles of the present invention may be employed.

In a first embodiment of the present invention illustrated in FIGS. 2 to 6, there is illustrated a communication module 40 that includes an antenna array 42, base plate 44 containing an antenna connector and activation reed housing (not shown). The communication module 40 further includes a multi-fit adaptor 46 for connection to a sensor array 14.

The antenna array 42 houses the radiating element and tuned ground planes, while the base plate 44 provides a connection port for the antenna and magnetic reed switch and activation key (PAK).

In one form a power activated key (PAK) may be used that comprises a magnetic activation switch, such that the communication module can be configured with the ability to be stored for an extended period of time, and when required can be activated by the magnetic switch. This will give the system extended shelf life allowing customers and distributors the ability to have components on hand that provide turnkey solutions which can be activated when located within an existing sensor network. The PAK may in one form be a solid-state magnetic switch that will activate the system or individual node when required. The system, once online may be switched off with a remote command and reactivated upon manual activation of the magnetic switch.

The interface housing and multi fit adaptor 46 allows for fitting to multiple existing sensor housings/access tubes that may or may not include an integrated battery. The present invention can be used with various sensors, such as EXO® sensors (Xylem Inc.), Dynagage sensors (Dynamax Inc.), thermal dissipation probe (TDP), sap velocity probes, soil moisture sensors or other environmental sensors.

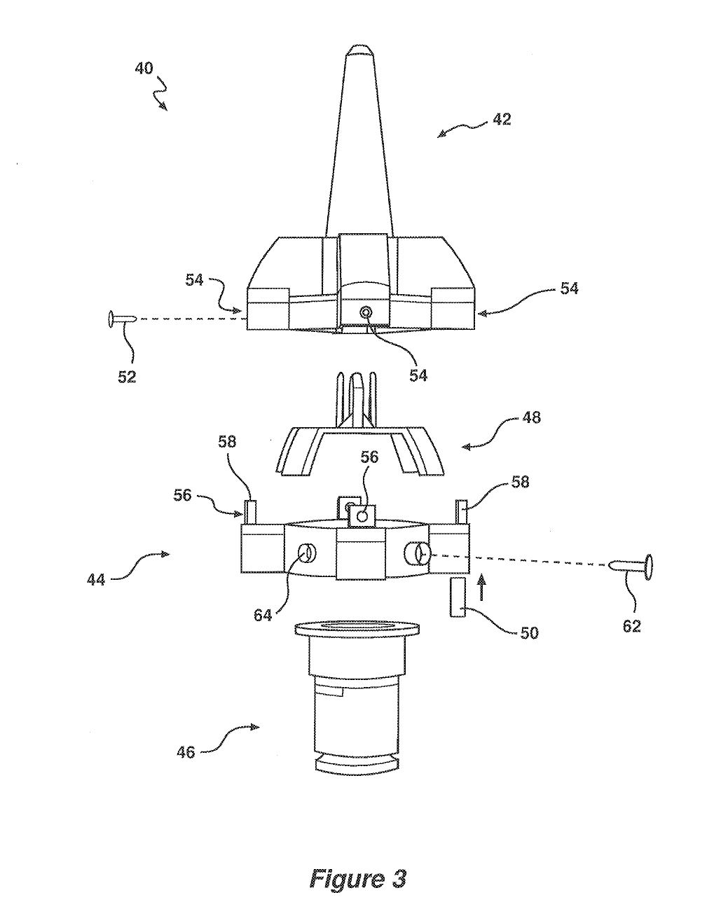

FIG. 3 illustrates an exploded view of the communication module 40, illustrating the antenna ground-plane shield 48 and activation port 50. The antenna ground plane shield 48 keeps ground plane and coax in place and secure. The activation port 50 slides into base plate 44 in the direction as indicated by the arrow in FIG. 3.

When the module 40 is assembled the antenna ground plane shield 48 is positioned within the antenna array 42. The antenna array 42 is attached to the base plate 44 by way of fixing members 52, such as screws, that engage through respective aperture 54 in the antenna array 42 and respective aperture 56 in tabs 58 that projected upwardly from the base plate 44. The skilled addressee will appreciate that not all the fixing means are illustrated in the figures, and other means of attachment are possible.

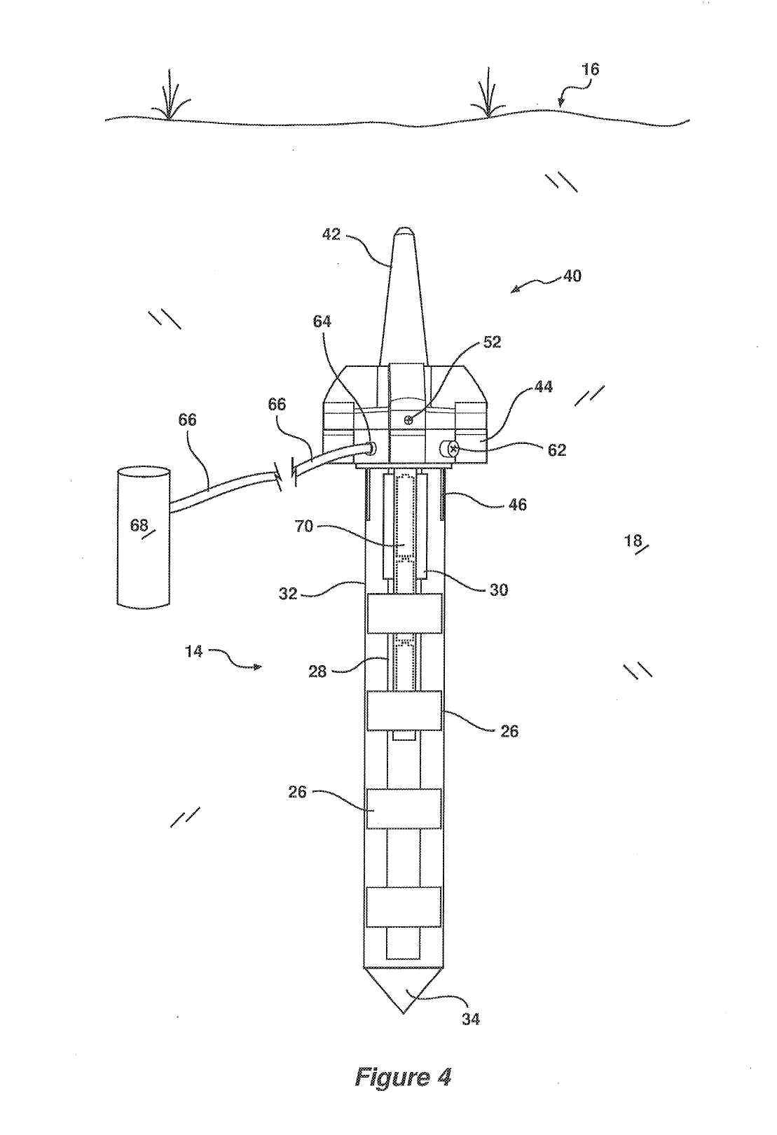

The base plate 44 includes aperture 60, through which is secured fixing means 62 to thereby attach the base plate 44 to the multi-fit adaptor 46. Furthermore, the base plate 44 may include an access port 64 to provide optional cable access. In some applications, the user may require external connection via cable 66 for power (AC or solar) or to connect a secondary sensor 68 to the communication module 40, as illustrated in FIG. 4. A cap (not shown) will be configured to close access port 64 when not in use.

As further illustrated in FIG. 4 the battery 70 of the present invention may be positioned within the access tube 32 of the sensor array 14 in the internal cavity produced by the generally annular sensors 26. The configuration of the communication module 40 means that once it is connected to the sensor array 14 it can be positioned in a subterranean arrangement, as illustrated in FIG. 4, or in an above ground arrangement.

The communication module 40 is configured for use in a wireless surface or subsurface network. Communication between the individual nodes 74 of the present invention is preferably wireless and may be selected from a group containing, but not limited to, radio-frequency (RF), WI-FI, BLUETOOTH®, near field communication or any other form of wireless communication means.

The data collected from the surface or subsurface network can be split at a base station so that the same data source can be used across multiple platforms. If the sensor network is connected to a soil moisture probe, the data can be collected and sent to an existing third party platform, such as IrriMAX® Live, by Sentek Pty Ltd. The same data can also be used to control pumps or valves based on preset conditions, as well as display current status locally.

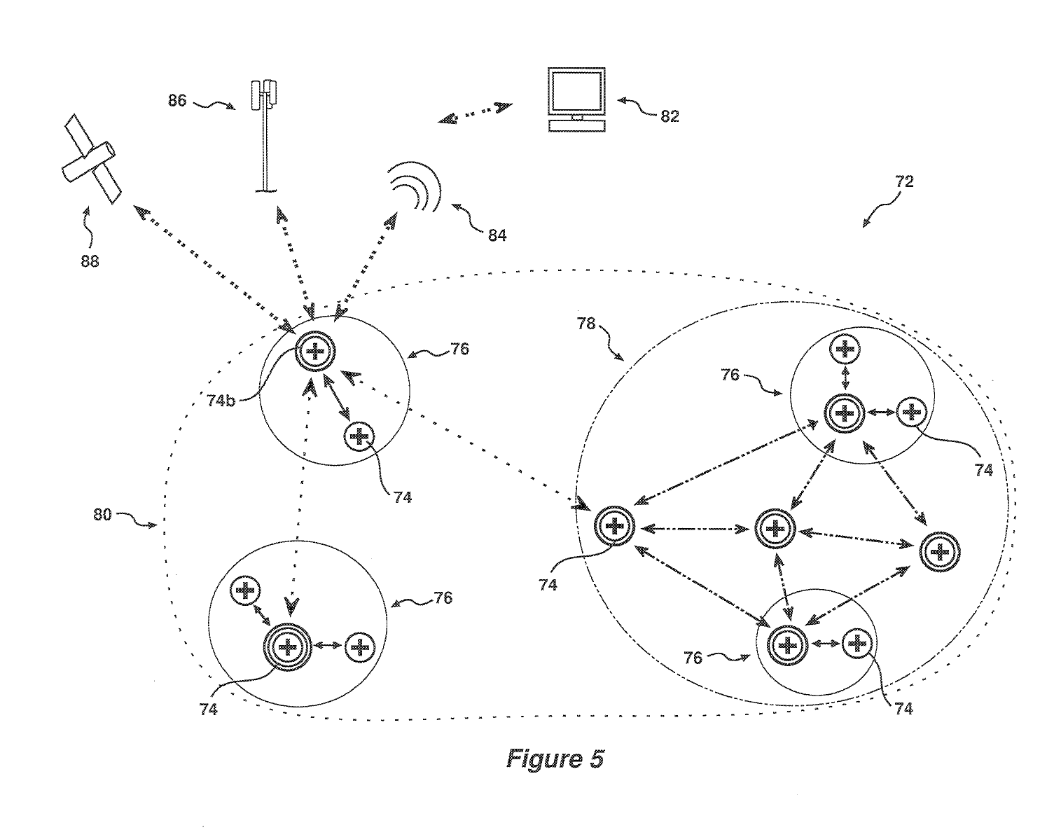

It is envisaged that the communication module 40 of the present invention will be used in a sensor network 72, such as the one illustrated in FIG. 5. The sensor network 72, may include a plurality of nodes 74 each comprising a communication module 40 and at least one sensor array 14. The nodes 74 are spread out across an environment and are able to communication which each other via a number of local area networks or sub-networks. In the present example, the networks or sub-networks include, surface or subsurface short range network/s 76 for communication between nodes 74 located in close proximity (10-100 m); medium range network/s 78 for communication between nodes 74 located at a distance from each other (100 m-1 km); and long range network/s 80 for communication between nodes 74 located at a large distance from each other (>1 km).

FIG. 5 illustrates communication with a CPU/GUI 82 by way of node 74b that is configured to act as a gateway unit or base station. The communication may be via a radio network 84 (i.e. between 900-930 MhZ or 915 MhZ) or other LAN, a telecommunication network 86 or a satellite network 88. A telecommunications network such as 3G/4G/5G could be used, or a modem communication path, network such as a local area network (LAN), or wide area network (WAN)/Internet could be used. Preferably security protocols and authentication will be used to ensure secure communication.

Each of the nodes 74 may also be BLUETOOTH® enabled or include hardware and corresponding software to enable short-range data transmissions (<10 m).

The reader should appreciate that the CPU/GUI 82 may include a number of components that are located at one site or at a number of sites, as is known in the art. For instance, the central processing unit may be located remote from the graphic user interface.

An example of a wireless network system that could be used includes a base to Near Line of Sight (NLS)—3.0 km Line of Sight (LOS), where there is some vegetation, wherein the signal is: 50 packets sent 50 received 100% @-60 db had a −50 db margin respectively.

Another example of a wireless network system that could be used includes a base to Near Line of Sight (NLS) Long Range (LR)—4.87 km Non-Line of Sight (NLS), where there is some vegetation and topography, wherein the signal is: 50 packets sent 40 received 80% @-85 db had a −30 db margin respectively.

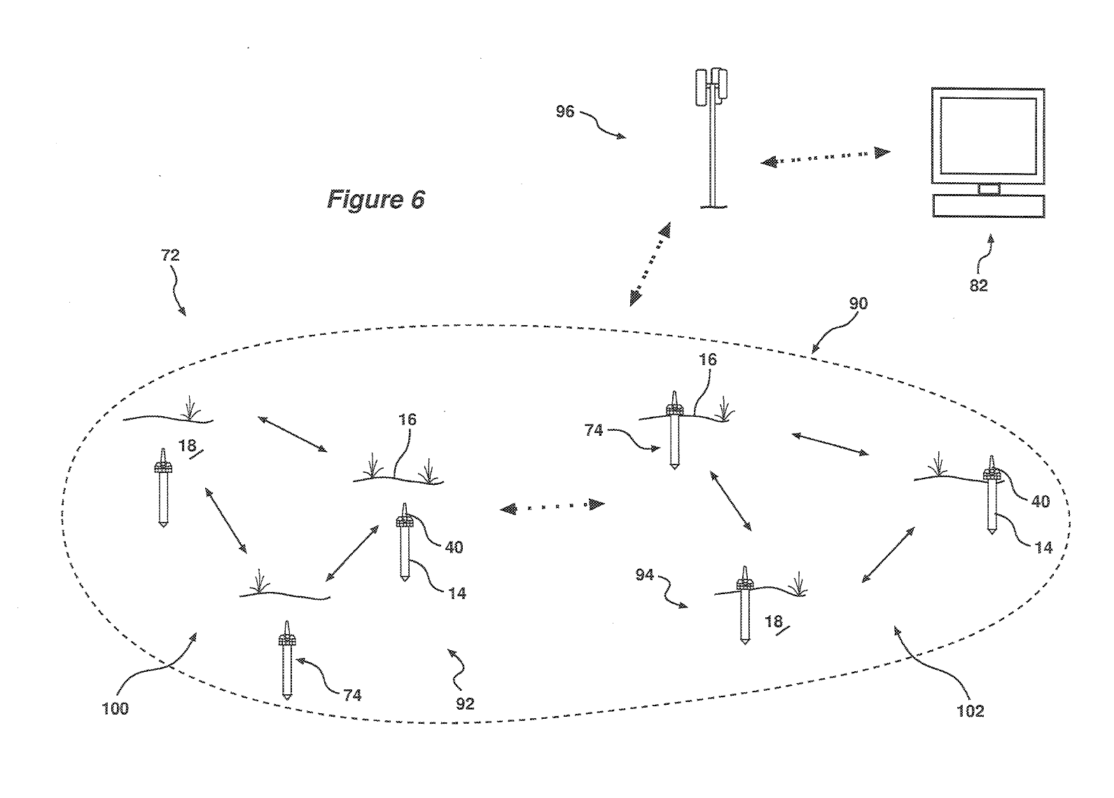

In another example as illustrated in FIG. 6 the sensor network 72 could be generally understood to comprise a designated local area network 90 comprising a plurality of interconnected nodes 74 that form a subsurface network 100 or a surface network 102, wherein the communication modules 40 are either positioned in a subterranean configuration 92 or in an above ground configuration 94. At least one of the nodes 74 within the sensor network 72 is configured to act as the gateway unit or base station wherein the local area network 90 is connected to the CPU/GUI 82 by way of a wide area network 96.

The sensor network 72 may include an onsite processor having specific application software that will be used to analyse information received from the networked sensors and forward the information to an off-site CPU/GUI 82 via a telecommunications network. The off-site CPU/GUI 82 is then able to further analyse the data and/or control other onsite systems such as, but not limited to irrigation systems.

The CPU/GUI 82 can alternatively be a local onsite system providing control regardless of current network status or availability, while also providing a basic redundancy for data display. An onsite processor may be connected to an onsite controller that is able to manage irrigation systems and/or other relevant systems in the event that the telecommunications network is unavailable. In another embodiment the CPU/GUI 82 may be located offsite or parts thereof and yet still permits the collection of data and control of onsite system such as irrigation systems.

Relevant application software may be stored in a computer readable medium such as electronic, magnetic, optical, electromagnetic, infrared, or semiconductor system, apparatus, system, device, propagation medium, or computer memory. In one possible embodiment, the system described herein includes hardware coupled to a microprocessor, microcontroller, System on Chip (“SOC”), or any other programmable device. The sensor network may include embedded software or firmware with corresponding hardware that is designed to perform one or more dedicated functions.

The system offers the ability for remote diagnostics so that basic functions, such as power levels, signal strength and system integrity can be monitored and logged. The power supply may be provided by primary cells and a modular design allows for multiple configurations to meet a range of applications, from 100 day vegetable crops, up to 6-8 years for playing fields or public areas.

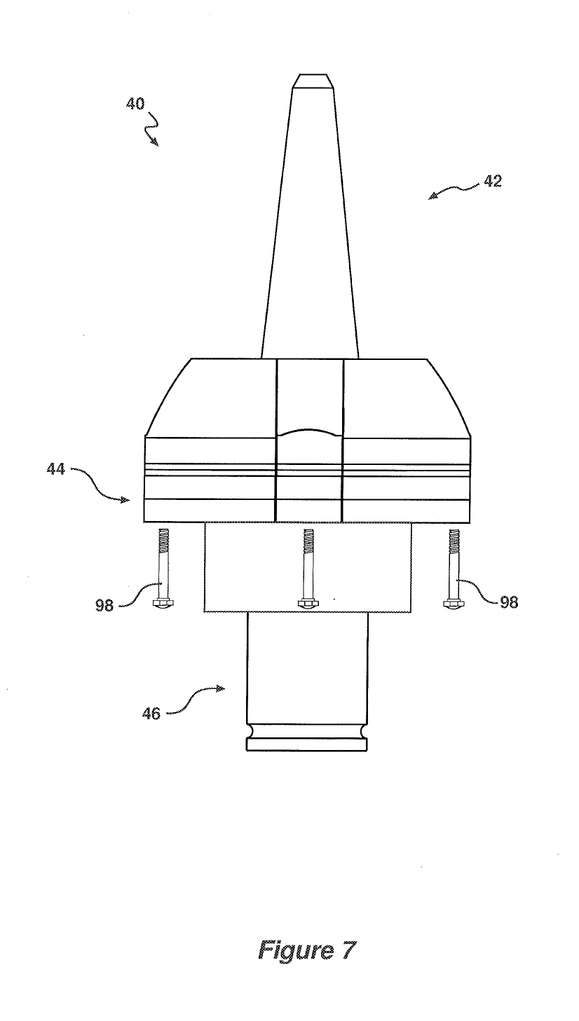

FIG. 7 illustrates another embodiment of the communication module 40 wherein the antenna array 42, base plate 44 and multi-fit adaptor 46 are connected by fixing means 98. It should however be appreciated by the reader that the elements of the communication module 40 may be integrally formed or include additional components such as seals or joining portions, without departing from the scope of the invention. For instance, the base plate 44 and multi-fit adaptor 46 may be integrally formed or the multi-fit adaptor 46 may be constructed from a number of detachable portions.

The illustrated invention provides advantages over the prior art. In various embodiments, the sensor network and communication module provides a number of benefits including integration with a range of existing sensors and platforms, multiple network options to maximize integration whilst reducing power requirements. In one form the invention provides a sensor network that is a surface/subsurface mesh, point to multi point, integrated self-learning sensor and control network. Some existing sensor networks are limited in their geographical range due to each sensor having to be in contact with a base unit. In contrast in some forms the present invention overcomes these issues by providing hierarchical networks and sub-networks that can effectively extend the geographical footprint of the overall system.

The communication module of the present invention provides a low profile discrete design, with a tuned ¼ wave antenna to maximize signal propagation through a range of mediums. The system allows multi use data, wherein data is utilised on multiple platforms to maximize system performance. The system allows access to data and control on local, remote or cloud based platforms.

The ingress protection rating allows the communication module, with sensor attached, to be used in subsurface installations. Accordingly, the sensor network provides the ability to operate in both surface and subsurface installations. The sensor network in one form provides a fully integrated surface and/or subsurface monitoring and control system.

Various features of the invention have been particularly shown and described in connection with the exemplified embodiments of the invention, however it must be understood that these particular arrangements merely illustrate the invention and it is not limited thereto. Accordingly, the invention can include various modifications, which fall within the spirit and scope of the invention.

1. A sensor network for measuring at least one parameter within a soil body, including:
at least one first node comprising a subsurface communication module for connection to a first sensor;
at least one second node comprising a surface communication module for connection to a second sensor;
wherein both of said subsurface communication module and said surface communication module are in communication with each other via at least one first network, and
wherein one of said subsurface communication module and said surface communication module acts as a gateway unit for communication with one of a computing device and a processor via a second network.

2. The sensor network according to claim 1 wherein the at least one first node comprises a plurality of first nodes and the at least one second node comprises a plurality of second nodes; wherein the first network comprises at least two sub-networks, wherein the plurality of first nodes create a subterranean sub-network and a plurality of second nodes create a surface sub-network.

3. The sensor network according to claim 1 or 2 wherein the first and second sensors measure one of the same parameter and different parameters, said parameter or parameters being one or more of soil moisture, nutrients, temperature, humidity, flow rates, pressure, voltage, current, salinity, and a condition within said soil body.

4. The sensor network according to claim 1 wherein the first network is a wireless local area network and the second network is wide area network.

5. The sensor network according to claim 4 wherein the first network is configured to connect the at least one first node and the at least one second node within from about 10 m up to about 100 m of each other by way of an independent low power radio network.

6. The sensor network according to claim 4 wherein the second network is one of GSM (Global System for Mobile communication), GPRS (General Packet Radio Service), Satellite, UMTS (Universal Mobile Telecommunications System), and CDMA (Code Division Multiple Access).

7. The sensor network according to claim 1, wherein each of the at least one first node and the at least one second node comprises a processor/interface, the associated one of the subsurface communication module and the surface communication module, and a power source.

8. The sensor network according to claim 7 wherein the processor/interface, the associated one of the subsurface communication module and the surface communication module, and the power source are positioned within a housing removably attached to a respective one of the first sensor and second sensor.

9. The sensor network according to claim 8 wherein one of the first node and the second node includes an auxiliary sensor for measuring a condition of said one of the first and the second sensor, or a condition of said associated one of the subsurface communication module and the surface communication module, or a condition of said power source.

10. The sensor network according to claim 1 wherein a secondary sensor is connected to one of the surface communication module and the subsurface communication module by way of a cable.

11. The sensor network according to claim 10 wherein said sensor network monitors a soil condition and control onsite systems, including one or more of irrigation pumps, valve controls, and generator controls.

12. The sensor network according to claim 11 wherein the surface communication module and subsurface communication module each include an antenna array having a radiating element and tuned ground planes.

13. A sensing node comprising a radio, an antenna, an interface, a power supply and one or more sensors, wherein the sensing node is locatable in one of a first position and a second position and, when in the first position, the sensing node is in a subterranean arrangement, and, when in the second position, a portion of the sensing node extends above a ground surface, whereby in either of said first and second positions, the sensing node is connectable to a respective wireless network.

14. A method of communication between sensors located within or adjacent a soil body and a computing device, including the steps of:
locating a plurality of first nodes in a soil body, each first node comprising a first communication module attached to a first sensor, wherein the respective first communication modules are concealed below a surface of said soil body;
locating a plurality of second nodes in the soil body, each second node comprising a second communication module attached to a second sensor, wherein at least a portion of the respective second communication modules extend above the surface of the soil body;
establishing a subsurface network between the plurality of first nodes;
establishing a surface network between the plurality of second nodes;
establishing a local area network between said first and second nodes;
establishing a wide area network between said plurality of first nodes and said plurality of second nodes and a computing device to transfer information from one or both of said plurality of first nodes and plurality of second nodes to said computing device.

15. The method according to claim 14 further including the step of controlling a control system based upon information received by said computing device from the one of said plurality of first nodes and said plurality of second nodes.
